(19) United States
(12) Patent Application Publication
(10) Pub. No.: US 2004/0053359 A1
(43) Pub. Date: Mar. 18, 2004

(54) Method for production of a multimeric protein by cell fusion

(76) Inventors: Thomas Ryll, Fremont, CA (US); Wenlin Zeng, Fremont, CA (US); John Babcook, Fremont, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/247,466

(22) Filed: Sep. 18, 2002

(51) Int. Cl. C12P 21/02; C12N 5/06; C07H 21/04; C12N 15/08; C07K 16/18

(52) U.S. Cl. 435/69.1; 435/320.1; 435/325; 530/388.1; 536/23.53; 435/451; 435/326

(57) ABSTRACT

The present invention features methods of producing a multimeric protein in a hybrid, recombinant cell, as well as for identifying recombinant cells expressing heavy chain which cells are useful in production of such hybrid cells. In the method of the invention, at least first and second recombinant cells are engineered to contain a first and second expression cassette, respectively, which expression cassettes contain an amplifiable marker and encode first and second components of the multimeric protein. The recombinant cells are then fused to produce a hybrid cell expressing the multimeric protein. The hybrid cell is then cultured to provide for amplification of the DNA encoding the first and second multimeric protein components. The methods of the invention are widely applicable to the production of proteins having two or more components.

### FIELD OF THE INVENTION

This invention relates generally to methods for use in gene expression and cell fusion techniques, particularly in the production of multi-component proteins.

### BACKGROUND OF THE INVENTION

Recombinant DNA techniques have been used for production of heterologous proteins in transformed host cells. Generally, the produced proteins are composed of a single amino acid chain or two chains cleaved from a single polypeptide chain. More recently, multichain proteins such as antibodies have been produced by transforming a single host cell with DNA sequences encoding each of the polypeptide chains and expressing the polypeptide chains in the transformed host cell (U.S. Pat. No. 4,816,397).

The basic immunoglobulin (Ig) structural unit in vertebrate systems is composed of two identical “light” polypeptide chains (approximately 23 kDa), and two identical “heavy” chains (approximately 53 to 70 kDa). The four chains are joined by disulfide bonds in a “Y” configuration, and the “tail” portions of the two heavy chains are bound by covalent disulfide linkages when the immunoglobulins are generated either by hybridomas or by B cells.

A schematic of the general antibody structure is shown in FIG. 1. The light and heavy chains are each composed of a variable region at the N-terminal end, and a constant region at the C-terminal end. In the light chain, the variable region (termed “V_{L}J_{L}”) is the product of the recombination of a VL gene to a JL gene. In the heavy chain, the variable region (V_{H}D_{H}J_{H}) is the product of recombination of first a D_{H} and a J_{H} gene, followed by a D_{H}J_{H} to V_{H} recombination. The V_{L}J_{L} and VHD_{H}J_{H} regions of the light and heavy chains, respectively, are associated at the tips of the Y to form the antibody's antigen binding domain and together determine antigen binding specificity.

The (C_{H}) region defines the antibody's isotype, i.e., its class or subclass. Antibodies of different isotypes differ significantly in their effector functions, such as the ability to activate complement, bind to specific receptors (Fc receptors) present on a wide variety of cell types, cross mucosal and placental barriers, and form polymers of the basic four-chain IgG molecule.

Antibodies are categorized into “classes” according to the C_{H} type utilized in the immunoglobulin molecule (IgM, IgG, IgD, IgE, or IgA). There are at least five types of C_{H} genes (Cμ, Cγ, Cδ, Cεc, and Cα), and some species (including humans) have multiple C_{H} subtypes (e.g., Cγ₁, Cγ₂, Cγ₃, and Cγ₄ in humans). There are a total of nine C_{H} genes in the haploid genome of humans, eight in mouse and rat, and several fewer in many other species. In contrast, there are normally only two types of light chain constant regions (CL), kappa (K) and lambda (λ), and only one of these constant regions is present in a single light chain protein (i.e., there is only one possible light chain constant region for every V_{L}J_{L} produced). Each heavy chain class can be associated with either of the light chain classes (e.g., a C_{Hγ}region can be present in the same antibody as either a κ or λ light chain).

A process for the immortalization of B cell clones producing antibodies of a single specificity involves fusing B cells from the spleen of an immunized mouse with immortal myeloma cells. Single clones of fused cells secreting the desired antibody could then be isolated by drug selection followed by immunoassay. These cells were given the name “hybridoma” and their antibody products termed “monoclonal antibodies.”

The use of monoclonal antibodies as therapeutic agents for human disease requires the ability to produce large quantities of the desired antibody. One approach to increased production was simply to scale up the culture of hybridoma cells. Although this approach is useful, it is limited to production of that antibody originally isolated from the mouse. In the case where a hybridoma cell produces a high affinity monoclonal antibody with the desired biological activity, but has a low production rate, the gene encoding the antibody can be isolated and transferred to a different cell with a high production rate.

In some cases it is desirable to retain the specificity of the original monoclonal antibody while altering some of its other properties. For example, a problem with using murine antibodies directly for human therapy is that antibodies produced in murine systems may be recognized as “foreign” proteins by the human immune system, eliciting a response against the antibodies. A human anti-murine antibody (HAMA) response results in antibody neutralization and clearance and/or potentially serious side-effects associated with the anti-antibody immune response. Such murine-derived antibodies thus have limited therapeutic value.

One approach to reducing the immunogenicity of murine antibodies is to replace the constant domains of the heavy and light chains with the corresponding human constant domains, thus generating human-murine chimeric antibodies. Chimeric antibodies are generally produced by cloning the antibody variable regions and/or constant regions, combining the cloned sequences into a single construct encoding all or a portion of a functional chimeric antibody having the desired variable and constant regions, introducing the construct into a cell capable of expressing antibodies, and selecting cells that stably express the chimeric antibody. Examples of methods using recombinant DNA techniques to produce chimeric antibodies are described in PCT Publication No. WO 86/01533 (Neuberger et al.), and in U.S. Pat. No. 4,816,567 (Cabilly et al.) and U.S. Pat. No. 5,202,238 (Fell et al.).

In another approach, complementarity determining region (CDR)-grafted humanized antibodies have been constructed by transplanting the antigen binding site, rather than the entire variable domain, from a rodent antibody into a human antibody. Transplantation of the hypervariable regions of an antigen-specific mouse antibody into a human heavy chain gene has been shown to result in an antibody retaining antigen-specificity with greatly reduced immunogenicity in humans (Riechmann et al. (1988) Nature 332:323-327; Caron et al. (1992) J. Exp. Med 176:1191-1195).

Another approach in the production of human antibodies has been the generation of human B cell hybridomas. Applications of human B cell hybridoma-produced monoclonal antibodies have promising potential in the treatment of cancer, microbial infections, B cell immunodeficiencies associated with abnormally low antibody production, and other diseases and disorders of the immune system. Obstacles remain in the development of such human monoclonal antibodies. For example, many human tumor antigens may not be immunogenic in humans and thus it may be difficult to isolate anti-tumor antigen antibody-producing human B cells for hybridoma fusion.

The present invention addresses these needs by providing a method for production of an antibody-expressing recombinant cell.

Literature

Patents and Published Applications

U.S. Patents

U.S. Pat. Nos. 5,916,771; 4,816,397; 4,816,567; 4,975,369; 5,202,238; 5,643,745; 5,683,899; 5,695,965.

Published Applications

EP 0 273 889; EP 0 088 994 B1; WO 86/01533; WO 92/15322; WO 93/19172; WO 93/25663; WO 94/02602; WO 95/02686; WO 95/30739; WO 98/16654.

Scientific Articles

Wood, et al., “High Level Synthesis of Immunoglobulins in Chinese Hamster Ovary Cells,” J. Immunol. 145(9):3011-3016 (Nov. 1, 1990).

Bebbington, C. R., (1991) “Expression of Antibody Genes in Nonlymphoid Mammalian Cells,” Methods: A Companion to Methods Enzymol. 2:136-145.

Bebbington, C. R., et al., (1992) “High-level Expression of a Recombinant Antibody from Myeloma Cells Using a Glutamine Synthetase Gene As An Amplifiable Selectable Marker,” Bio/Technology 10: 169-175.

Caron, P. C., et al., (1992) “Engineered Humanized Dimeric Forms of IgG Are More Effective Antibodies,” J. Exp. Med. 176:1191-1195.

Cattaneo, A. and Neuberger, M. S., (1987) “Polymeric Immunoglobulin M Is Secreted by Transfectants of Non-lymphoid Cells in the Absence of Immunoglobulin J Chain,” J. 6:2753-2758.

Cockett, M. I., et al., (1990) “High-level Expression of Tissue Inhibitor of Metalloproteinases in Chinese Hamster Ovary Cells Using Glutamine Synthetase Game Amplification,” Bio/Technology 8:662-667.

Graham, F. L., and Vander Eb, A. J., (1973) “A New Technique for the Assay of Infectivity of Human Adenovirus 5 DNA,” Virology 52:456-467.

Riechmann, L. et al., (1988) “Reshaping Human Antibodies for Therapy,” Nature 332:323-327.

Kranenborg, et al., “Development and Characterization of Anti-Renal Cell Carcinoma X Antichelate Bispecific Monoclonal Antibodies for Two-Phase Targeting of Renal Carcinoma,” Cancer Research (December 1995), vol. 55, No. 23, pp. 5864S-5867S.

Cao, et al., “A Rapid Non-Selective Method to Generate Quadromas by Microelectrofusion,” J. Immunol. Methods (Nov. 16, 1995), vol. 187, No. 1, pp. 1-7.

Salazar-Kish, et al., “Comparison of a Quadroma and its Parent Hybridomas in Fed Batch Culture,” J. Biotechnology (1993), vol. 30, No. 3, pp. 351-365.

Bos, et al., “Enhanced Transfusion of a Bacterial Plasmid of Hybrid Hybridoma Quadroma Cell) Lines,” Hybridoma (February 1992), vol. 11, No. 1, pp. 41-51.

### SUMMARY OF THE INVENTION

The present invention features methods of producing a multimeric protein in a hybrid, recombinant cell, as well as for identifying recombinant cells expressing heavy chain which cells are useful in production of such hybrid cells. In the method of the invention, at least first and second recombinant cells are engineered to contain a first and second expression cassette, respectively, which expression cassettes contain an amplifiable marker and encode first and second components of the multimeric protein. The recombinant cells are then fused to produce a hybrid cell expressing the multimeric protein. The hybrid cell is then cultured to provide for amplification of the DNA encoding the first and second multimeric protein components. The methods of the invention are widely applicable to the production of proteins having two or more components.

In one embodiment, the multimeric protein is an antibody composed of antigen-specific heavy and light chains. A first DNA construct encoding the desired heavy chain (or a fragment of the heavy chain) and a first amplifiable marker is introduced into a first mammalian host cell to produce a first recombinant cell, while a second DNA construct encoding the desired light chain (or a fragment of the light chain) and a second amplifiable marker is introduced into a second mammalian host cell to produce a second recombinant cell. In one embodiment, where amplification is performed only after fusion as described below, the first and second amplifiable markers are the same, i.e., are amplified by a single amplification agent. The first and second constructs can also include a marker which allows for selection or screening of the first and second recombinant cells to identify those cells containing the construct prior to fusion. Preferably, the marker on the first construct is different from the marker on the second construct.

The first recombinant host cell and the second recombinant host cell are then combined by cell fusion to form a third cell, referred to herein as the hybrid cell. Prior to fusion of the first and second cells, the transformed cells may be selected for specifically desired characteristics, e.g., high levels of expression. The first and second cells subjected to fusion can be either single cell clones or pools of first and second cells.

After fusion, the resulting hybrid cell contains and expresses both the DNA encoding the desired heavy chain and the DNA encoding the desired light chain, resulting in production of the multimeric antibody. After screening or selecting for desired expression characteristics, the hybrid cell is then subjected to gene amplification by culturing the hybrid cell in the presence of the amplification agent. Where amplification is performed only after fusion (e.g., and not both before and after fusion), the amplifiable marker is preferably the same for both the heavy and light chain constructs. Where the amplifiable marker for the heavy chain and light chain constructs are the same, culturing in the presence of a single amplification agent provides for amplification of both the heavy chain- and light chain-encoding DNA, thus providing for increased expression of both chains in the hybrid, amplified cell.

In another aspect, where the multimeric protein is an antibody, the invention features a method for screening for a first recombinant mammalian cells expressing a heavy chain prior to fusion with a second recombinant mammalian cell expressing a light chain. While conventional wisdom indicated that efficient heavy chain expression and secretion requires co-expression of a light chain (e.g., such as an irrelevant light chain), the inventors have surprisingly found that certain mammalian cells that have been modified to contain a heavy chain-encoding construct express and secrete the encoded heavy chain into the culture supernatant, even in the absence of light chain expression. Thus, the invention further contemplates methods of identification of heavy chain-expressing and -secreting mammalian cells by detection of heavy chain in culture supernatant, e.g., using ELISA. This aspect of the invention provides for screening of large number of heavy chain-expressing clones that is readily adaptable to large-scale, high-throughput procedures.

One advantage of the invention is that amplification need only be performed after fusion of the first and second recombinant cells, and not both before and after fusion (e.g., the method does not require both amplification of each of the first and second recombinant cells prior to fusion (“pre-fusion amplification”), and amplification of the hybrid cell after fusion (“post-fusion amplification”).

Another advantage of the invention is that, where the amplifiable marker is the same in each of the first and second expression cassettes encoding the multimeric protein components, the hybrid cell need only be cultured in the presence of a single amplification agent (e.g., in contrast to systems that use different amplifiable markers for the light chain construct and for the heavy chain construct). This both simplifies the production process, making scale-up less cumbersome, and avoids the need to subject the hybrid cell to the harsh conditions that can be associated with use of multiple amplification agents.

An important advantage of the invention is that, as the inventors have discovered, amplification of the hybrid, fused cell is relatively easier, less time and resource consuming, and more efficient than separate amplification of each the parental recombinant heavy chain-expressing and recombinant light chain-expressing cells prior to fusion. Without being held to theory, this is may be due to the toxic effect of over-amplified heavy chain on the parental heavy chain-expressing cell.

Another advantage of the invention is that cells expressing a single component of the final multi-component protein can be individually selected for one or more desired characteristics, such as a high rate of production, optimal expression, fast growth rate, stable expression, and the like. Furthermore, this selection occurs prior to fusion of cells forming the hybrid cell and prior to production of the final multimeric protein, thus increasing the likelihood of production of a hybrid cell having desired characteristics.

Another advantage is that the method generates a cell which produces an antibody at a high rate through the fusion of two kinds of cells which are each selected prior to fusion for high production of the desired heavy or light chains.

Another advantage is that the final multi-component protein is not expressed until all the cells expressing the individual components of the multi-component protein are fused into a single hybrid cell.

Other aspects, features, and advantages of the invention will become apparent from the following detailed description, and the claims.

### BRIEF DESCRIPTION OF THE FIGURES

FIG. 1 is a schematic showing the basic immunoglobulin structure.

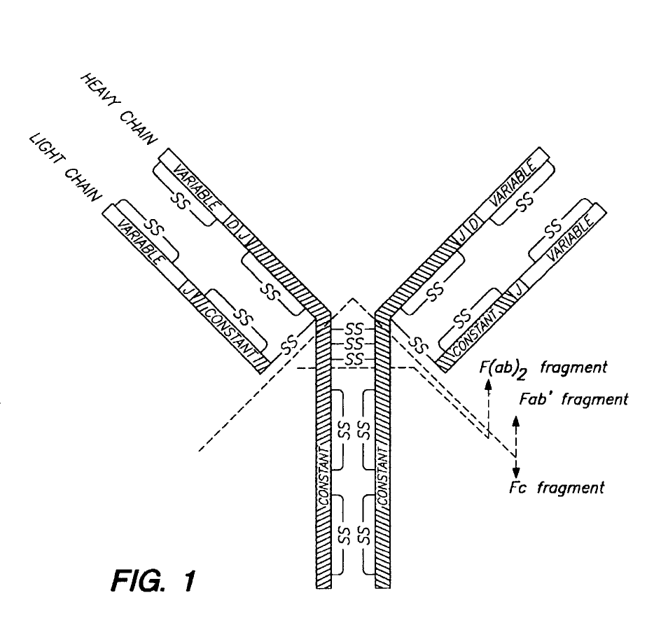

FIG. 2 is a flow chart showing a generic scheme of production first and second transformed host cells and fusion to produce a hybrid cell expressing the multimeric protein, exemplified in this figure by an antibody. (AM=amplifiable marker; HC=heavy chain; LC=light chain).

FIG. 3 is a schematic illustrating a specific embodiment of the invention in which DHLFR⁻ CHO cells are independently transfected with (i) pPS1-HC-Gamma, a human heavy chain Ig construct and (ii) pPS1-LC-Kappa, a human light chain Ig construct. The independent cell lines are selected and fused to yield a hybrid cell containing the human heavy chain Ig construct and the human light chain Ig construct. The hybrid cell is then subjected to amplification using an agent suitable for use with the amplifiable marker, exemplified by DHLFR with the amplification agent methotrexate (MTX).

FIG. 4 is graph showing the correlation between heavy chain as detected by ELISA of culture supernatant (Y axis) and of cell lysates (X axis) of recombinant CHO cells.

FIG. 5 is a table summarizing antibody titers in culture supernatants before and after amplification. The CCF1 protocol refers to use of pools of heavy chain- and light chain-expressing recombinant cells to produce fused hybrid cells. The CCF2 protocol refers to use of single cell clones of heavy chain- and light chain-expressing recombinant cells to produce fused hybrid cells.

FIG. 6 is a table summarizing heavy chain (HC) and light chain (LC) RNA level before and after amplification, demonstrating that HC and LC genes are amplified using a single amplification marker and a single amplification agent.

FIG. 7 is a graph showing the stability of MTX-amplified clones using the CCF1 protocol (amplification post-fusion) or CCF3 protocol (amplification of HC-expressing and LC-expressing cells prior to fusion). Both protocols produce stable expression cell lines.

Before the methods and compositions of the present invention are described and disclosed it is to be understood that this invention is not limited to the particular methods and compositions described as such may, of course, vary. It is also to be understood that the terminology used herein is for the purpose of describing particular embodiments only, and is not intended to be limiting since the scope of the present invention will be limited only by the appended claims.

It must be noted that as used in this specification and the appended claims, the singular forms “a”, “an” and “the” include plural references unless the context clearly dictates otherwise. Thus, for example, reference to “a DNA sequence” includes a plurality of DNA sequences and different types of DNA sequences.

Unless defined otherwise all technical and scientific terms used herein have the same meaning as commonly understood by one of ordinary skill in the art to which this invention belongs. Although any materials or methods similar or equivalent to those described herein can be used in the practice or testing of the present invention, the preferred methods and materials are now described.

All publications mentioned herein are incorporated herein by reference for the purpose of describing and disclosing the particular information for which the publication was cited. The publications discussed above are provided solely for their disclosure prior to the filing date of the present application. Nothing herein is to be construed as an admission that the inventor is not entitled to antedate such disclosure by virtue of prior invention.

### DETAILED DESCRIPTION DEFINITIONS

Unless defined otherwise, all technical and scientific terms used herein have the same meaning as commonly understood by those of ordinary skill in the art to which this invention pertains. Any methods and materials similar or equivalent to those described herein can be used in the practice or testing of the present invention, the described methods and materials being exemplary.

The terms “polynucleotide”, “nucleic acid”, and “nucleotide sequence” are used interchangeable herein to mean any polynucleotide, e.g., RNA or DNA, usually DNA, or nucleic acid fragment of interest which may be introduced into a cell, including an intact gene or fragment of a gene. When the method of the invention is used to generate an antibody, the nucleotide sequence of interest will be all or part of either the constant region and/or variable region of the light or heavy chains, and may include all, part, or none of the regulatory nucleotide sequences that control expression of the light or heavy chain.

The nucleotide sequence of interest for heavy chains includes but is not limited to all or a portion of the V, D, J, and switch regions (including intervening sequences) and flanking sequences. For light chains, the nucleotide sequence of interest includes but is not limited to the V and J regions, and flanking and intervening sequences. Of particular interest are heavy chain- and light chain-encoding polynucleotides that are “rearranged” (i.e., relative to unrearranged genomic IFN-γ-encoding sequence) so that, when expressed together in a suitable host cell, the polynucleotides provide for expression of an antibody, e.g., an antibody that binds an antigen of interest.

The polynucleotide may be naturally occurring, synthetic, or partially natural and partially synthetic. The polynucleotide may also be a non-naturally occurring or modified naturally-occurring sequence. The polynucleotide can include sequences taken from different sources, e.g., different species. For example, when the method is used to produce an antibody, the DNA may encode a chimeric (for example, human-mouse) immunoglobulin chain, or it may be a CDR-grafted DNA sequence having a human immunoglobulin sequence with antigen-specific murine CDR sequences. The DNA may encode a fully human antibody. B-cells obtained from non-human animals immunized with an antigen and also hybridoma, trioma, and quadromas derived from such B-cells can also provide the nucleotide sequence introduced into the host cells. B-cells and hybridomas producing any kind of monoclonal antibody may be used as a source of the nucleotide sequence, including cells producing, for example, fully mouse monoclonal antibodies, fully human monoclonal antibodies, CDR-grafted monoclonal antibodies, chimeric monoclonal antibodies, and F(ab)₂.

By the terms “multi-component”, “multichain”, or “multimeric” protein is meant a protein composed of two or more proteins or polypeptides. The method of the invention is useful for producing a multimeric protein by the fusion of two or more cells each expressing a single component of the multimeric protein. For example, in one embodiment the multi-component protein is an antibody generated from two heavy chains encoded by DNA transfected into a first cell and two light chains encoded by DNA transfected into a second cell, where the final multimeric antibody is produced by a hybrid cell formed from the fusion of the first and second cells. “Multi-component,” “multichain,” and “multimeric” protein is meant to include any heterodimeric or hetero-oligomeric protein (e.g., BMP2/BMP7 heterodimeric osteogenic protein, ICE (interleukin-1 converting protein), receptors of the nucleus (e.g., retinoid receptors), heterodimeric cell surface receptors (e.g., T cell receptors), integrins (e.g, cell adhesion molecules, β₁-integrins, (see, e.g., Hynes, 1987 Cell 48:549-554; Hynes 1992 Cell 60:11-25), tumor necrosis factor (TNF) receptor, and soluble and membrane-bound forms of class I and class II MHC (major histocompatibility complex proteins). Where the multimeric protein is a receptor, “multimeric protein” is meant to encompass soluble and membrane forms of the receptor.

By the term “introducing” a polynucleotide into a cell means inserting an exogenous piece of DNA into a cell, including but not limited to transfection or transduction with a vector, such that all or part of the exogenous nucleotide sequence is stably maintained in the cell, and the resulting transformed cell expresses the introduced nucleotide sequence.

By “transformed” or “transfected” is meant that the host cell is modified to contain an exogenous polynucleotide, which can be chromosomally integrated or maintained in the cell as an episomal element. These terms are used interchangeably herein without limitation as to the method of introduction of the exogenous polynucleotide, unless specifically noted otherwise.

By the term “fusing” or “fusion” of two or more cells is meant a method in which two or more cells are combined to form a single hybrid cell which contains all or part of at least the nucleic acid content of each individual cell. Fusion may be accomplished by any method of combining cells under fuseogenic conditions well known in the art (See, for example, Harlow & Lane (1988) in Antibodies, Cold Spring Harbor Press, New York). Known methods for fusing cells includes by use with polyethylene glycol (PEG) or Sendai virus.

By the term “hybrid cell” is meant a cell formed by combining two or more cells, e.g., by fusion. In the method of the invention, hybrid cells are formed from the fusion of one or more transformed cells each expressing a single component of a multimeric protein.

The term “amplification” as used herein refers to the increase or replication of an isolated region within a cell's DNA. Amplification is achieved using an amplification agent that is selected for amplification of a particular amplifiable marker. Thus “amplification system” refers to such compatible amplification agents and markers (e.g., the amplification agent methotrexate (MTX) and the amplifiable marker dihydrofolate reductase (DHFR)). Amplification or the making of successive copies of the amplifiable marker results in greater amounts of the product of the amplifiable marker being produced in the face of greater amounts of amplification agent. Amplification pressure is applied even in the presence of an endogenous gene corresponding to the amplifiable marker (e.g., endogenous DHFR), by adding ever greater amounts of the amplification agent to the media.

The term “irrelevant” as in, e.g., “an irrelevant light chain” means a light chain which does not contribute to the binding of the antigen of interest and is not a component of the multimeric protein produced by the hybrid cell of the invention.

By the term “desired” component, e.g., desired heavy chain, or desired light chain, is meant an immunoglobulin chain which recognizes the antigen of interest.

Generation of a Hybrid Cell Producing a Heterologous Multimeric Protein

The present invention is based on the discovery of advantageous methods for the production of a multimeric protein in a hybrid, recombinant cell that is the product of at least two parental cells expressing different components of the multimeric protein from a construct having an amplifiable marker. The inventors have found that amplification of the component-expressing constructs in the hybrid cell after fusion is more efficient than amplification of the construct in each of the parental cells prior to fusion since it consumes less time and resources. Furthermore, the inventors have discovered that amplification of both constructs occurs even when the same amplifiable marker is used on each of the component-encoding constructs. This discovery allows for the use of a single amplification agent, thus avoiding the additional stress an additional selection agent imposes upon the hybrid cell and simplifying the production process, particularly in the context of commercial scale-up. The invention thus provides an improved method for production of hybrid cells expressing a multimeric protein of interest.

The inventors have also made the surprising discovery that certain mammalian cells, e.g., Chinese hamster ovary (CHO) cells, express and secrete heavy chain polypeptide from a heavy chain-encoding construct even in the absence of the expression of a light chain polypeptide. This discovery can be applied to the methods of the invention to facilitate identification of a heavy chain-expressing parental cell prior to fusion, e.g., using ELISA of cell culture supernatants or other method, particular those that can be readily adapted to high throughput assay systems.

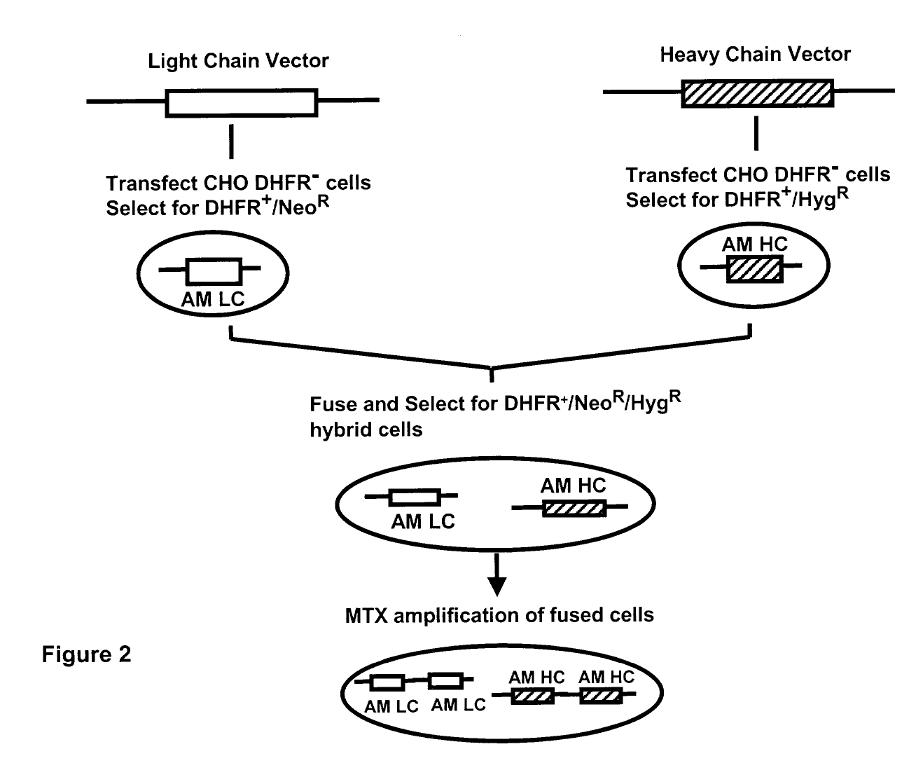

The present invention thus provides methods for generating a hybrid cell producing a multi-component protein from two or more recombinant cells, each of which cells expresses at least one component of the multimeric protein from a construct comprising an amplifiable marker. In general, as illustrated schematically in FIG. 2, the method comprises: 1) introducing a first polynucleotide into a first mammalian cell, wherein the first polynucleotide comprises a first amplifiable marker, a sequence encoding a first component of the multi-component protein (exemplified in FIG. 2 by a heavy chain polypeptide), and may also include a first selectable or detectable marker, wherein the first amplifiable marker is amplifiable by an amplification agent; 2) introducing a second polynucleotide into a second mammalian cell, wherein the second polynucleotide comprises a second amplifiable marker, a sequence encoding a second component of the multi-component protein (exemplified in FIG. 2 by a light chain polypeptide), and may also include a second selectable or detectable marker; 3) optionally, repeating step 2) for each remaining component of the multi-component protein; 4) fusing cells produced from steps 1)-3) to form a hybrid cell, wherein the hybrid cell expresses the multi-component protein; and 5) culturing the hybrid cell in the presence of the amplification agent for the first and second amplifiable markers. The multi-component protein (e.g., antibody) can then be recovered from the hybrid cell culture. Where amplification is to be performed both pre- and post-fusion, the first and second amplification markers may be different or the same (i.e., amplifiable by different or the same amplification agent, respectively).

In one embodiment, the recombinant parental cells having the first or second component-encoding constructs, respectively, are not subjected to amplification prior to fusion to produce a hybrid cell, e.g., the parental recombinant cells are not cultured in the presence of an amount of the amplification agent(s) sufficient to provide for amplification of the amplifiable marker prior to fusion.

In one embodiment of particular interest, the second amplifiable marker is amplifiable by the same amplification agent as the first amplifiable marker. The selectable or detectable markers are preferably different for each construct for each component of the multi-component protein. In this embodiment, amplification may be performed either before or after fusion, preferably after fusion of the recombinant parental cells.

In specific embodiments, the multi-component protein is an antibody, and the first and second components are a heavy chain polypeptide and a light chain polypeptide, respectively.

In more specific embodiments, the method of the invention can be conducted according to one of the following exemplary protocols.

Protocol 1 (Also Referred to Herein as CCF1)

Select a pool of cells expressing heavy chain (HC) from a first construct having HC-encoding DNA and having a first selection marker and an amplifiable marker associated with the HC-encoding DNA

Select a pool of cells expressing light chain (LC) from a second construct having LC-encoding DNA and having a second selection marker and an amplifiable marker associated with the LC-encoding DNA, where the amplifiable markers of the HC and LC constructs are amplifiable by the same amplification agent

Fuse the HC and LC pools of cells to produce and select hybrid cells using both selection markers of both of the HC and LC constructs

Obtain single cell clones of hybrid cells

Screen the hybrid cells for the best clones (i.e., clones having desired characteristics)

Subject hybrid cells to amplification of the amplifiable markers of the HC and LC constructs using a single amplification agent

Obtain pools and single cell clones of amplified hybrid cells

Screen the amplified hybrid cells for best clones (i.e., clones having desired characteristics)

Protocol 2 (Also Referred to Herein as CCF2)

Select a clone expressing a heavy chain (HC) and having desired characteristics, where the clone expresses the HC from a first construct having HC-encoding DNA, a first selection marker, and an amplifiable marker associated with the HC-encoding DNA

Select a clone expressing a light chain (LC) and having desired characteristics, where the clone expresses the LC from a second construct having a second selection marker and an amplifiable marker associated with the LC-encoding DNA

Fuse the HC and LC clones to produce and select hybrid cells using both selection markers

Obtain single cell clones of hybrid cells

Screen the hybrid cells for the best clones (i.e., clones having desired characteristics)

Subject hybrid cells to amplification of the amplifiable markers

Obtain pools and single cell clones of amplified hybrid cells

Screen the amplified hybrid cells for best clones (i.e., clones having desired characteristics)

In each of Protocols 1 and 2, the amplifiable marker of each HC- and LC-encoding DNAs (e.g., DHFR) are amplified by a single amplification agent (e.g., methotrextate)

Generation of a cell expressing the desired heavy chain can be accomplished using methods available in the art. For example, production of a heavy chain-expressing cell can involve: (1) identifying and cloning and/or synthesizing the gene, gene fragment, or nucleotide sequence encoding the variable segment or antigen-binding sequences of the heavy chain. The nucleotide sequence may be obtained from either a cDNA or genomic source, or synthesized de novo and is preferably rearranged (i.e., the genomic segments of the immunoglobulin locus encoding the antigen-binding segment of the heavy chain are operably linked so as to encode an antigen-binding segment that binds a preselected antigen); (2) cloning the nucleotide sequence encoding the desired constant regions of the heavy chain; (3) ligating the variable region with the constant region so that the complete nucleotide sequence can be transcribed and translated to express the desired heavy chain polypeptide; (4) ligating the construct into a vector containing a selectable marker and appropriate gene control regions; (5) amplifying the construct in bacteria; (6) introducing the vector into eukaryotic cells; (7) selecting the cells expressing the selectable marker; and (8) screening the cell supernatants or lysates for the expressed heavy chain. Similarly, a cell expressing a desired light chain construct can produced according to methods known in the art, including a method as outlined above for the heavy chain construct.

Alternatively, the process of generating a cell expressing a desired heavy or light chain may involve (1) construction of a Ig chain DNA sequence containing (a) a signal sequence, (b) the gene, gene fragment, or nucleotide sequence encoding the variable region or antigen-binding sequences, and (c) the nucleotide sequence encoding the desired constant region of the Ig chain, followed by (2) PCR amplification of the Ig construction, (3) insertion of the construct into eukaryotic cells, (4) selecting the cells expressing the selectable marker, and (5) screening the cells for the expressed Ig chain. Optionally, the cells expressing the desired heavy chain or the desired light chain can be further selected for desirable characteristics, such as heavy or light chain production rate or level, ability of the expressed heavy or light chain to combine with another light or heavy chain, respectively, to provide an antibody having a desired antigen binding affinity, and/or other characteristics desirable for heavy or light chain production or function in an antibody.

Transformed cells expressing or capable of expressing the desired component of the multimeric protein are fused by methods known in the art to form a hybrid cell expressing the multimeric protein. When the multimeric protein is an antibody, the DNA sequences encoding the desired immunoglobulin may be composed entirely of sequences originating from a single species, e.g., fully human or fully murine, or may be contain sequences originating from more than one species, e.g., a human-mouse chimera or CDR-grafted humanized antibody. The hybrid cell produced antibody product may also contain a desired antigen binding site (variable region) linked to a desired constant region. Thus, a specifically designed antibody may be generated with a desired antigenicity combined with the desired isotype.

Prior art methods for independently expressing the light and heavy chains in a single host cell are known, see, e.g,. U.S. Pat. No. 4,816,397, European patent application publication No. 88,994, PCT published patent application WO 93/19172, U.S. Pat. No. 4,816,567, U.S. Pat. No. 4,975,369, U.S. Pat. No. 5,202,238, PCT published patent application WO 86/01533, PCT published patent application WO 94/02602, and European published patent application No. 273,889.

Vector Constructs

The constructs useful in the methods of the invention are generally recombinant DNA vectors including, but not limited to, plasmids, phages, phagemids, cosmids, viruses, retroviruses, and the like, which insert a nucleotide sequence into a cell.

Methods for introducing an exogenous nucleotide sequence of interest into a cell, including a suitable mammalian cell, are known in the art. These methods typically include use of a DNA vector to introduce the nucleotide sequence into the genome or a cell or cells, and then growing the cells to generate a suitable population.

In general, polynucleotides are introduced into mammalian cells by lipofection (e.g., using Lipofectamine (Gibco)), electroporation, CaPO₄ precipitation, RBC ghost fusion, protoplast fusion, and the like (see, e.g., Neumann et. al. (1982) EMBO J. 1:841; Felgner et. al. (1987) PNAS 84:7413. Lipofection is particularly preferred when the host cells are CHO cells.

The polynucleotide encoding a component of the desired multi-component protein may be obtained as a cDNA or as a genomic DNA sequence by methods known in the art. For example, messenger RNA coding for a desired component may be isolated from a suitable source employing standard techniques of RNA isolation, and the use of oligo-dT cellulose chromatography to segregate the poly-A mRNA. When the product multi-component protein is an antibody, suitable sources of desired nucleotide sequences may be isolated from mature B cells or a hybridoma culture. Where the multi-component protein is an antibody, the components include a heavy chain and a light chain polypeptide. The components may be of any desired origin (e.g., mammalian, particularly human, mouse, rat, goat, rabbit, sheep, cow, horse and the like preferably human, including chimeras) and may, upon production, exhibit binding to any antigen or epitope of interest.

The construct further includes an amplifiable marker which is increased in expression, and providing an increased expression of flanking DNA (e.g., the adjacent heavy chain or light chain-encoding polynucleotide) when the cell is cultured in the presence of an amplification agent. An amplifiable marker and an amplification agent pair are referred to herein as an “amplification system.” Suitable amplification systems for use in the invention include, without limitation, dihydrofolate reductase (DHFR) and methotrexate (MTX); glutamine synthase (GS) and Methionine Sulphoximine (MSX) (see, e.g, Bebbington, C. R., et al. 1992. Bio/Technology 10: 169-175), and Adenosine deaminase (ADA) and 2′-deoxycoformycin (dCF) (Kaufman et al. 1986. Proc. Natl. Acad. Sci. USA. 83:3136-3140). Other amplification systems suitable for use in the invention are described in, for example, Ausubel et al. 1989. Current Protocols in Molecular Biology. John Wiley & Sons, New York). The DHFR-MTX gene amplification system is a preferred gene amplification system for use in the methods of the invention, particularly where the host cells are CHO cells, particularly DHFR-deficient (DHFR-) CHO cells.

In addition to the nucleotide sequence encoding the desired component of the product multi-component protein, vector constructs can include additional components to facilitate replication in prokaryotic and/or eukaryotic cells, integration of the construct into a eukaryotic chromosome, and markers to aid in selection of and/or screening for cells containing the construct (e.g., the detectable markers and drug resistance genes discussed above for the targeting construct). For eukaryotic expression, the construct should preferably additionally contain a promoter sequence positioned 5′ of the gene to be expressed and a polyadenylation sequence positioned 3′ of the gene to be expressed. The promoter sequence may be selected from any of a variety of promoter sequences known in the art. The polyadenylation signal sequence may be selected from any of a variety of polyadenylation signal sequences known in the art. Preferably, the polyadenylation signal sequence is the SV40 early polyadenylation signal sequence.

Host Cells

Host cells may include any mammalian cell or cell line, capable of expressing the desired component of the multimeric protein, and amenable to fusion to form a hybrid cell suitable for expression of the multi-component protein. For example, where the desired protein is an antibody, the cell line is any mammalian cell capable of expressing a functional antibody. Nonlymphoid cell lines are of particular interest (Cattaneo & Neuberger (1987) EMBO J. 6:2753-2758; Deans et al. (1984) Proc. Natl. Acad. Sci. 81:1292-1296; Weidle et al. (1987) Gene 51:21-29). The ability of nonlymphoid cell lines to assemble and secrete fully functional antibodies may be exploited for antibody production. For example, Chinese hamster ovary (CHO) cells and their DHFR negative variants DG44 and DUXB11 (Urlaub, G et al., Cell, 33: 405-412 (1983), Urlaub, G et al., Somatic Cell and Molec. Gent., 12: 555-566 (1986), Urlaub, G and Chasin, LA, Proc. Natl. Acad. Sci., 77: 4216-4220, (1980), Graf, LH and Chasin, LA, Mol. Cell. Biol., 2: 93-96 (1982))., Chinese Hamster lung (V79) cells, and COS cells have well-characterized efficient expression systems and have been used for both long-term and transient expression of a variety of proteins (Bebbington (1991) supra; Rauschenbach et. al. (1995) Eur. J. Pharm. 293:183-90). A method for achieving a high level of expression of DNA sequences encoding a chimeric antibody in transformed NSO myeloma cells has been described (Bebbington et al. (1992) Bio/Technology 10:169-175). DHFR negative CHO cells are of particular interest.

Mammalian myeloma cells also find use as host cells in the methods of the invention, including non-secreting (NS) myeloma cells (e.g., a non-secreting (NSO) myeloma). Other myeloma cells include mouse derived P3/X63—Ag8.653, P3/NS1/1-Ag4-1(NS-1), P3/X63Ag8.U1(P3U1), SP2/0—Ag14 (Sp2/0, Sp2), PAI, F0, and BW5147; rat derived 210RCY3—Ag.2.3; and human derived U-266AR1, GM1500-6TG-A1-2, UC729, CEM-AGR, DIR11, and CEM-T15.

Selection of Transformed Cells

Detection of recombinant host cells, which contain the desired constructs, integrated as desired, can be accomplished in a number of ways, depending on the nature of the introduced polynucleotide. If the introduced polynucleotide includes a selectable marker, the initial screening of the transfected cells is to select those which express the marker. Any of a variety of selectable markers known in the art may be included, such as guanosine phosphoryl transferase gene (gpt), neomycin resistance gene (Neo), hygromycin resistance gene (Hyg) and hypoxanthine phosphoribosyl transferase (HPRT). For example, when using a drug resistance gene, those recombinant host cells that grow in the selection media containing the drug (which is lethal to cells that do not contain the drug resistance gene) can be identified in the initial screening. It will be appreciated that a variety of other positive, as well as negative (i.e., HSV-TK, cytosine deaminase, and the like), selectable markers that are well known in the art can be utilized in accordance with the present invention for selection of specific cells and transfection or other events. As well, a variety of other marker genes (i.e., the LacZ reporter gene and the like) can be utilized in similar manners.

After a period of time sufficient to allow selection to occur the surviving cells are then subjected to a second screening to identify those recombinant cells which express the desired peptide component of interest. This may be accomplished by, for instance, an immunoassay using antibodies specific for the particular immunoglobulin class.

The protocol for the second screening depends upon the nature of the polypeptide encoded by the inserted polynucleotide and, in some instances, the nature of the host cell. For example, where the recombinant cell contains a polynucleotide that, when expressed, does not produce a secreted product, selection or screening for the presence of cells having the introduced polynucleotide can be accomplished by Southern blot using a portion of the exogenous polynucleotide sequence as a probe, or by polymerase chain reaction (PCR) using sequences derived from the exogenous polynucleotide sequence as probe. The recombinant cells containing the desired polynucleotide sequence can also be identified by detecting expression of a functional product, e.g., immuno-detection of the product. Alternatively, the expression product can be detected using a bioassay to test for a particular effector function conferred by the exogenous sequence.

Where the first host cell is transfected with DNA encoding heavy chain, the expression of the heavy chain can be tested using any conventional immunological screening method known in the art, for example, ELISA conducted with cell lysate samples (see, for example, Colcher et al. Protein Engineering 1987 1:499-505). The cell can be further selected for additional desirable characteristics such as heavy chain production rate or level, ability of the expressed heavy chain to combine with light chain to provide an antibody of a desired antigen binding affinity, and other characteristics desirable for heavy chain production and heavy chain function in an antibody.

Nonlymphoid cells expressing a desired protein may be transfected in a number of ways known to the art. For example, a first CHO cell may be transfected with a vector comprising a DNA sequence encoding a desired light chain and a second CHO cell transfected with a vector comprising a DNA sequence encoding a desired heavy chain. Transfected cells are selected and fused. Fused cells are selected for expression of an antibody having the desired light chain Ig and heavy chain Ig.

Prior to the present invention, detection of heavy chain expression in a non-lymphoid cell, was met with the technical issue that the heavy chain polypeptide was not secreted at a detectable level in the absence of light chain expression. Failure of a cell to secrete the heavy chain peptide may make detection of transfectants more difficult since it necessitates assaying the cells themselves (e.g., by Northern blot analysis or immunodetection), as opposed to conveniently screening the cell supernatant by ELISA. One means for avoiding this issue is to fuse a heavy chain encoding cell (as evidenced by such cell's survival of selection for a selectable marker provided in the heavy chin-encoding construct) with a cell expressing a light chain, and detect successful fusion products expressing the antibody, e.g., by ELISA. However, this approach does not ensure that the CHO cell containing the heavy chain-encoding sequence expresses the heavy chain polypeptide at a desirable level.

Another method for addressing this issue is to provide for expression of an irrelevant light chain in the host cell containing the heavy chain construct. Co-expression of the irrelevant light allows for expression and secretion of the Ig heavy chain. The gene encoding the irrelevant light chain may either be integrated into a chromosome or be present in an episomal vector, such as bovine papilloma virus (BPV) or other episomal vector known in the art. After selection for transformants, expression of the heavy chain is easily confirmed by an ELISA assay of the cell lysates for secreted antibody. During the fusion process, random chromosomes are normally lost. Thus, it is expected that cells lacking the irrelevant Ig light chain will be generated during the fusion process. These hybrid cells can easily be identified by ELISA assay of the supernatants for the presence of the desired chains and absence of the irrelevant chain.

Thus, in one embodiment, the desired light chain of the final antibody product is the κ light chain. In such cases, an Igλ expressing myeloma cell is transfected with the desired heavy chain (IgH) gene. After transfection with a plasmid carrying the desired heavy chain and selection, cells expressing the heavy chain are examined directly for expression of the desired heavy chain, e.g., ELISA assay of the supernatants with antibody specific to the heavy chain. The second cell, e.g., a non-secreting myeloma cell, is transfected with a desired light chain (IgL), and transfectants detected through e.g., Northern blot analysis or immuno-detection with an antibody specific to the κ light chain. The cells expressing the light chain can be further selected for desirable characteristics associated with production of a functional light chain, such as light chain production rate or level, ability of the expressed light chain to combine with heavy chain to provide an antibody of a desired antigen binding affinity, and other characteristics desirable for light chain production and heavy chain function in an antibody. After fusion, the hybrid cell expressing the desired IgH/IgL antibody is selected for the presence of the kappa light chain and desired heavy chain (e.g., C_{gamma1}, C_{gamma2}, C_{gamma3}, C_{gamma4}, and the like) and the absence of lambda light chain, e.g., by ELISA assay of the culture medium.

In a specific embodiment of the invention, the inventors have surprisingly found that certain mammalian cells containing a heavy chain-expressing construct secrete the heavy chain polypeptide into the culture medium even when the cell does not express a light chain polypeptide. Without being held to any particular theory or mode of operation, mammalian cells that lack accessory proteins that prevent or inhibit secretion of heavy chain polypeptide from the cell are suitable for use in the methods of the invention. For example, the inventors have found that CHO cells and CHO derivative cells (e.g., DHFR⁻ variants) secrete heavy chain polypeptide in the culture supernatant. Preliminary evidence indicates that, in the inventors' hands and under the culture conditions used, heavy chain polypeptide was not detectable in the culture supernatant of HEK 293 cells. Thus heavy chain-expressing cells can be chosen for use in subsequent fusion by detection of heavy chain polypeptide in the culture supernatant, thus facilitating rapid identification and providing for adaptation to commercial scale-up and high-throughput techniques. This approach facilitates identification of a heavy chain-expressing host cell having desired characteristics for use in fusion with a light chain-expressing host cell fusion partner. This approach also avoids the need to identify hybrid cells that do not express a light chain polypeptide, and avoids the need to introduce an irrelevant light chain into the host cell that is to express the desired heavy chain.

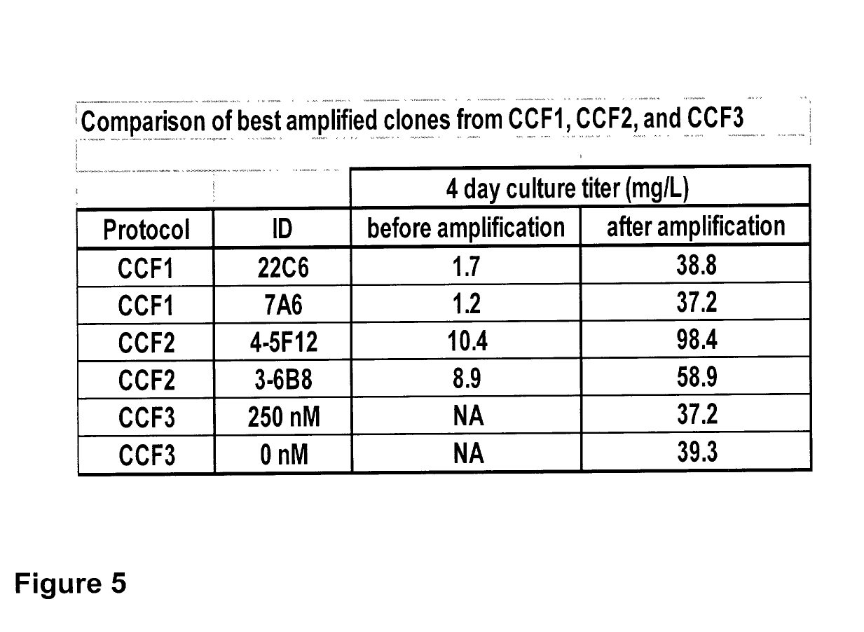

The level of heavy chain polypeptide secretion need not be high, but rather only correlate reasonably well with total heavy chain polypeptide produced in the cell (e.g., both secreted and non-secreted). For example, as illustrated in FIG. 5, the level of heavy chain (HC) secreted in the supernatant is not very high compared to HC that remained inside the cell (supernatant (sup) vs. lysate). However, because the level of HC in the supernatant corresponds reasonably well with the level of HC in whole cell lysates, identification of HC-expressing cells for further manipulation by screening supernatant is as efficient way to select desirable HC-expressing cells from a large number of cells prior to fusion.

Once suitable fusion partners are identified, a first recombinant cell expressing the desired heavy chain and a second recombinant cell expressing the desired light chain are then fused under appropriate fuseogenic conditions according to methods well known in the art (see, e.g., Harlow & Lane, supra). Any combination of cells capable of expressing a desired heavy chain or desired light chain and that can be fused to produce a hybrid cell expressing both heavy and light chains can be used. Thus, the first cell (e.g., expressing the desired heavy chain) can be of the same or different type as the second cell (e.g, expressing the desired light chain), e.g., the first cell can be a myeloma cell and the second cell can be a non-lymphoid cell. Preferably, the first and second cells are of the same type, more preferably CHO cells. The first and second recombinant cells can be selected and cultured as single cell clones prior to fusion, or can be fused as pools of cells.

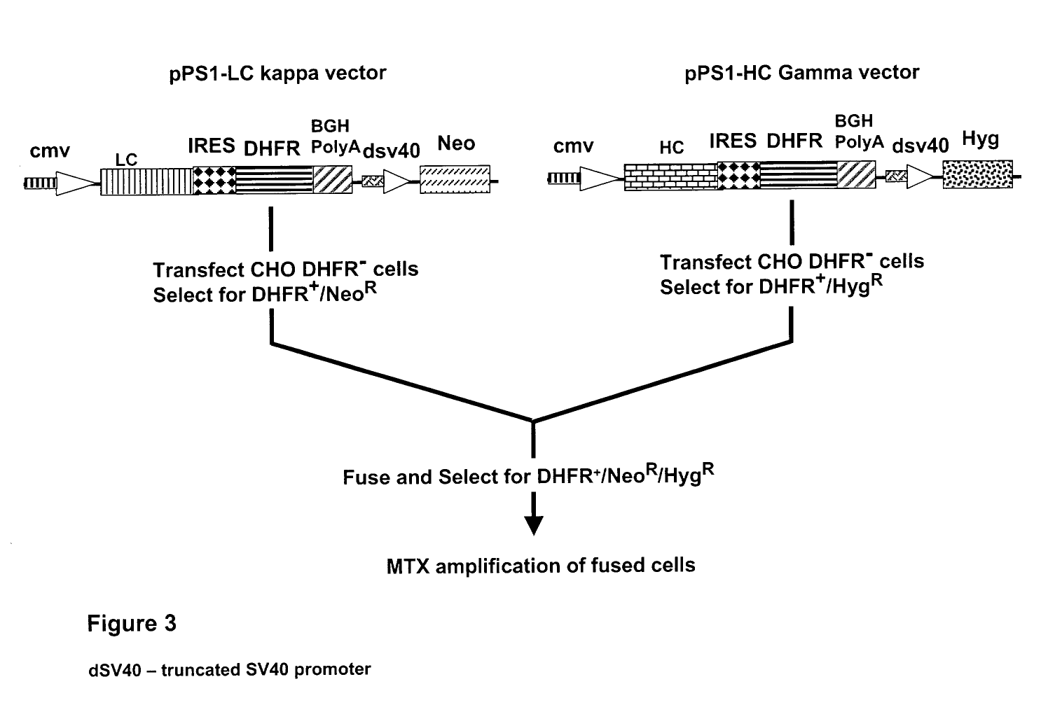

In another embodiment of the invention, selection of fused or hybrid cells can be initially determined through the utilization of distinct marker genes in each of the “parental” cells or cell lines. An example of this approach is illustrated in FIG. 3. There, a parental CHO cell line, that is DHFR-, is transfected with a vector (pPS1-LC-Kappa) that contains the DHFR gene and the neomycin resistance gene (Neo). Another parental CHO cell line, that is DHFR-, is transfected with a vector (pPS1-HC-Gamma) that contains the DHFR gene and the hygromycin (Hyg) resistance gene. The DHFR genes in this embodiment also serve as an amplifiable marker. In this example, the DHFR gene is positioned 3′ of the heavy chain- or light chain-encoding region on the construct and an internal ribosome entry site (IRES) positioned in-between. Each cell line, following transfection, contains distinct selectable markers (i.e., hygromycin resistance in the first and neomycin/G418 resistance in the second). Thus, upon fusion, resulting “daughter” cells in which fusion has been successful will be resistant to both hygromycin and G418. The screening technique of the invention is advantageous in that it mitigates the need to determine expression of immunoglobulin molecules in order to determine if a fusion has been successfully performed. Following fusion, the hybrid cell is then subjected to amplification using methotrexate (MTX).

Under certain fusion conditions, cells and cell lines can become spontaneously resistant to G418, and, possibly, other selectable markers. Thus, in certain embodiments of the invention, it is preferable to utilize selectable markers to which cells and cell lines are less likely to spontaneously generate resistance. An example of one such marker is the hypoxanthine phosphoribosyl transferase gene (HPRT) which confers resistance to hypoxanthine aminopterin. Another marker that can be used in tandem with HPRT resistance is the LacZ gene. The LacZ gene is not a selectable marker; but, rather, acts as a marker gene which, when expressed by a cell, stains blue in the presence of β-galactosidase.

In other embodiments, the parental cell lines may be produced from different cells, and these different cells fused to form hybrid cells, which can then be subjected to amplification. For example, a first parental cell line, which is HPRT deficient (such as the CHO, P3X, NSO, and NS0-bc12 myeloma cell lines), is transfected with a light chain or heavy chain expression cassette. The cassette can include, for example, appropriate antibody genes, an amplifiable marker (AM), and an HPRT selectable marker. Transfected cells of the first parental cell can be selected through HPRT selection and cells producing high levels of antibodies can be picked. A second parental cell line (such as the CHO, P3X, NS0, and NS0-bc12 myeloma cell lines), which is also preferably HPRT deficient, is transfected with an light chain or heavy chain expression cassette. The cassette includes, for example, appropriate coding sequence, an amplifiable marker, (AM) and the LacZ gene. Transfected cells of the second parental cell can be selected through staining with β-gal. As will be appreciated, either the first or second parental cell line can include the light chain genes or the heavy chain genes and the other of the first or second parental cell line will contain the other of the light or heavy chain genes. As will also be appreciated, other selectable markers can be included in the cassettes utilized to transfect the cells. Upon fusion of the first and second parental cell lines, successful fusion can be determined through HPRT selection and β-gal staining of daughter cells. Daughter cells can be further selected based upon expression levels of immunoglobulin molecules.

For example, in specific embodiments, a first parental cell line, exemplified by the myeloma cell line, NS0, which is HPRT deficient, is transfected with a light chain cassette containing a gene amplification system (AM), a human light chain gene system (VKJKCK), and an HPRT selectable marker (HPRT). A second parental cell line, exemplified by any one of J558L, Ag.1, or NS0, are transfected with a heavy chain cassette containing a gene amplification system (AM), a human antibody heavy chain gene system (V_{H}D_{H}J_{H}hγ), and the LacZ gene. In this embodiment, AM in each of the heavy chain and light chain constructs is amplifiable by the same amplification agent (e.g., AM in each of the constructs is DHFR). Success of transfection can be determined through the use of the selectable marker HPRT and through β-gal staining. Additionally cells can be picked for expression of light chain or heavy chain.

Following isolation and generation of parental cell lines incorporating the antibody gene cassettes, fusion between a parental cell line including heavy chain genes and a parental cell line including light chain genes is conducted. Such fused cells can be readily identified through dual marker selection, that is, HPRT selection and β-gal staining. Cells which have been successfully fused, will be HPRT resistant and will stain positive with β-gal. Following identification of desired hybrid cells, the hybrid cells are then subjected to amplification by culturing in the presence of the suitable amplification agent (e.g., where AM is DHFR, a suitable amplification agent is methotrexate).

### EXAMPLES

The following examples are put forth so as to provide those of ordinary skill in the art with a complete disclosure and description of how to make and use various constructs and perform the various methods of the present invention and are not intended to limit the scope of what the inventors regard as their invention. Unless indicated otherwise, parts are parts by weight, temperature is in degrees centigrade, and pressure is at or near atmospheric pressure. Efforts have been made to ensure accuracy with respect to numbers used, (e.g., length of DNA sequences, molecular weights, amounts, particular components, etc.) but some deviations should be accounted for.

### EXAMPLE

### Generation of Hybrid Cells Containing Light and Heavy IG Chains Using Protocols CCF1 and CCF2

The following example illustrates the application of the methods of the invention based on cell-to-cell fusion with post-fusion gene amplification where the parental cells are provided as either pools of cells expressing the desired heavy or light chain (the “CCF 1” protocol) or single cell clones of cells expressing the desired heavy or light chain (the “CCF2” protocol).

Transfection of Chinese Hamster Ovary (CHO) Cells:

6×10⁶ of DHFR deficient CHO cells (DG44, Graf, LH and Chasin, LA, Mol. Cell. Biol., 2:93-96 (1982); G. Urlaub, E. Kas, A. M. Carothers, and L. A. Chasin, Cell, 33:405-412 (1983)) were transfected with 30 mcg of linearized heavy chain (HC) vector or light chain (LC) vector using Lipofectamine 2000 following the protocol from the manufacturer. In the pPS1-HC-Gamma and pPS1-LC-Kappa vectors (illustrated in FIG. 3), both HC and LC are controlled by the CMV promoter and are linked to the amplification marker DHFR via internal ribosomal entry site (IRES). Linking the HC and LC gene with DHFR through IRES improves co-amplification of the HC and LC gene along with the DHFR gene; however, such linkage is not required for co-amplification of HC and LC with DHFR. The HC and LC vector transfected cells were cultured a 10 cm dish in the growth media [CHO—S SFM II, 5% FBS and 1× GHT (110 μM hypoxanthine, 41 μM thymidine and 133 μM glycine)] for 48 hours.

Selection of Stable Transfectants:

After 48 hours of culturing in growth media, cells transfected with the light chain vector were cultured in the Neomycin and DHFR selection media (CHO-S SFM II without hypoxanthine and thymidine, 5% dialyzed FBS, and 450 μg/ml Neomycin). Stable single clones of transfectant CHO cells were selected by plating the transfected cells in 96 well plates using limited dilution method and culturing them in the Neo and DHFR selection media.

Stable pools of transfectants were selected by culturing the transfected cells en mass in T75 flasks in the selection media.

Stable single clones and pools of heavy chain transfectant CHO cells were selected with the selection media containing 350 μg/ml hygromycin following the above described procedures.

Selection of Stable Heavy Chain (HC) and Light Chain (LC) Transfectant Clones That Express High Titer of Heavy Chain Peptide and Light Chain Peptide:

Light chain expression level of single LC transfectant clones in 96 well plates were initially determined by ELISA analysis of the light chain peptide secreted in the supernatant. Clones that showed high level of light chain secretion were further expanded to T flasks and their light chain peptide expression level was further determined by a 4-day culture supernatant ELISA assay. 2×10⁵ cells of each clone were cultured in 1 ml of selection media in 24 wells for 4 days. Light chain peptide titer in the supernatant was analyzed with a light chain specific Elisa assay. Clones that have the highest light chain titer were expanded in culture and selected for fusion.

Identification of CHO cells secreting HC in the absence of LC

As discussed above, conventional wisdom indicated that in the absence of expression of a light chain polypeptide, heavy chain polypeptide is not efficiently secreted from recombinant cells. As a result, expression of heavy chain in HC transfectant mammalian cells were usually determined by ELISA or western blot analysis of heavy chain protein in cell lysate. Cell lysates preparation is labor intensive and low through-put, as it is difficult to screen large number of HC transfectant clones in a 96 well format using these cumbersome techniques.

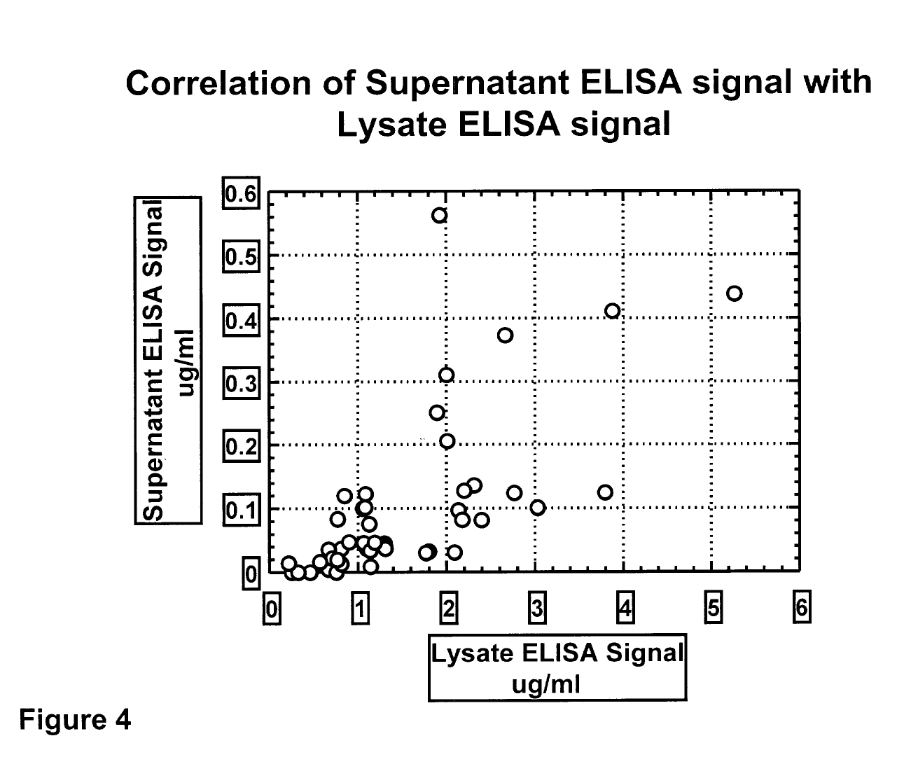

Surprisingly, the inventors found that heavy chain is secreted into the culture supernatants of HC transfectant CHO cells. Heavy chain polypeptide was detected in the supernatant using a heavy chain-specific ELISA assay. Comparison of the heavy chain level in both supernatant and cell lysates of about 40 HC transfectant clones showed that the level of the heavy chain polypeptide secreted into the supernatant correlate reasonably well with heavy chain protein inside the cells as determined by ELISA assay of the cell lysates (FIG. 4). Therefore, Elisa analysis of the supernatant of HC transfectant CHO clones can be used in a high-through-put 96 well plate format to screen large number of HC transfectant clones and identify the clones that express high level of heavy chain.

As a result, heavy chain expression level of the single HC transfectant clones in 96 well plates were also initially determined by ELISA analysis of the heavy chain peptide secreted into the culture supernatant. Clones that showed high level of heavy chain secretion were selected and expanded further. The expression level of the clones were further determined and ranked by Elisa analysis of the culture supernatant as well as the cell lysates of a 4-day culture as described above. The best clones that gave the highest expression level in both supernatant and lysate were further expanded and selected for fusion.

Generation of a Hybrid Cell Expressing an Antibody by Fusing Stable Pools of Heavy Chain and Light Chain Transfectant Cells (Protocol CCF1):

Stable pools of heavy chain transfectant cells and stable pools of light chain transfectant cells were washed separately with 50 ml of warm CHO-S SFM II media twice. 10×10⁶ of heavy chain transfectant cells and 10×10⁶ of light chain transfectant cells were combined in a 50 ml conical tube, and warm CHO-S SFM II added to bring the volume to 50 ml. The cells were then centrifuged at 1000 RPM for 5 minutes. All CHO-S SFM II media was aspirated from the cell pellets. The conical tube was placed in a 37° C. water in a beaker so that the cell pellet is under the water level. 1 ml of warm PEG was slowly added to the cell pellet over one minute, and the pellet gently stirred into the PEG for one minute. 2 ml of warm CHO-S SFM II was then stirred in over two minutes, followed by 8 ml of warm CHO-S SFM II stirred in over three minutes. The fused cells were then centrifuged to remove all PEG/CHO-S SFM II solution, and the pellet was resuspended in 10 ml of the fusion selection media (CHO-S SFM II without hypoxanthine & thymidine, 5% dialyzed FBS, 450 μg/ml of Neo and 350 μg/ml of Hyg) by inverting the tubes gently.

Single clones of hybrid cells that contain both heavy chain and light chain vectors were selected by plating the PEG fused cells at appropriate cell densities in 96 well in the fusion selection media described above until colonies appeared. Expression of the full antibody by the fused cells was detected by analyzing the supernatant with an ELISA that detects both the light chain and heavy chain of the antibody. Clones of hybrid cells that secreted high titer of antibody as identified by ELISA were expanded further in fusion selection media. Antibody titers of the clones were further determined by a 4-day culture supernatant ELISA assay as the following: 2×10⁵ cells of each clone were cultured in 1 ml of fusion selection media in 24w for 4 days. Antibody titer in the supernatant was analyzed with ELISA (FIG. 5).

Hybrid cell clones with high antibody expression were expanded and selected for DHFR amplification.

Generation of a Hybrid Cell Expressing an Antibody by Fusing Cells Producing High Titer of Heavy Chain and Cells Secreting High Titer of Light Chain (Protocol CCF2):

Cells from eight clones that exhibited the highest heavy chain expression and cells from four clones that exhibited the highest light chain expression were fused with PEG in a procedure similar to that described above for fusion of stable pools with the following modification. Equal number of cells from each of the eight HC transfectant clones were combined, and equal number of cells from each of the four LC transfectant clones were combined. 5×10⁶, 10×10⁶, 20×10⁶, and 40×10⁶ each of the HC and LC transfectant cells were fused with PEG as described above. After fusion, single cell clones of hybrid cells were selected by plating cells at appropriate density in 96w plates in the fusion selection media (CHO-S SFM II without hypoxanthine & thymidine, 5% dialyzed FBS, 450 μg/ml Neo, and 350 μg/ml Hyg). Stable pool of hybrid cells were selected by culturing cells in T flasks in the same fusion selection media.

Antibody titer of the pools and single clones of hybrid cells were measured with an ELISA assay that detects both the heavy chain and light chain. The antibody production level of the pools and single cell clones of hybrid cells generated by fusing high HC and LC transfectant cells are higher than those of the hybrid cells generated by fusing stable pools of HC and LC transfectant cells (FIG. 5).

Hybrid cell clones with high antibody expression were expanded and selected for DHFR amplification.

DHFR Amplification of Hybrid Cells:

Methotrexate (MTX) mediated DHFR gene amplification has been used widely to increase the expression of protein that is linked to the DHFR amplification marker (Ausubel et al. 1989. Current Protocols in Molecular Biology. John Wiley & Sons, New York). When heavy chain and light chain genes are co-transfected into the cells on either two separate plasmids or on one plasmid, it is very likely that both heavy chain and light chain genes will be co-amplified since both genes will be closely linked. However, in the present case of hybrid cells, the heavy chain vector and light chain vector are located on two different chromosomes. Thus it was not clear whether both genes would be amplified using MTX mediated DHFR amplification. Moreover, one way to avoid this problem is to use two different amplification markers, one for heavy chain and one for light chain, to ensure that both genes were amplified in hybrid cells (see, e.g., Wood et al. 1990. J. Immunol. 145(9):3011-3016). However, the two-marker system means subjecting the hybrid cells to two amplification treatments, which is harsh to the cells. In addition, the use of two amplification agents requires fine-tuning of the two amplification pressures in order to achieve amplification of both genes to compatible degrees.

Thus, the inventors set out to determine whether both the heavy and light chain genes could be successfully amplified using only a single amplifiable marker, which in this case was DHFR. Several clones of hybrid cells (derived from CCF1 and CCF2 protocols described above) secreting the high level of antibody were cultured in CHO-S SFM II, 5% dialyzed FBS, 450 μg/ml Neo, 350 μg/ml Hyg containing various concentrations of methotrexate (50 nM to 2 μM) in either 24 well or 6 well plates or T25 flasks with media exchange about once every few days until colonies appeared. Cells that survived the MTX amplification were further expanded to T75 flasks in the MTX containing media. Antibody titer of hybrid cells amplified with each MTX concentration was determined by ELISA analysis of a 4-day cell culture supernatant as described above for ranking the expression level of the amplified hybrid clones.

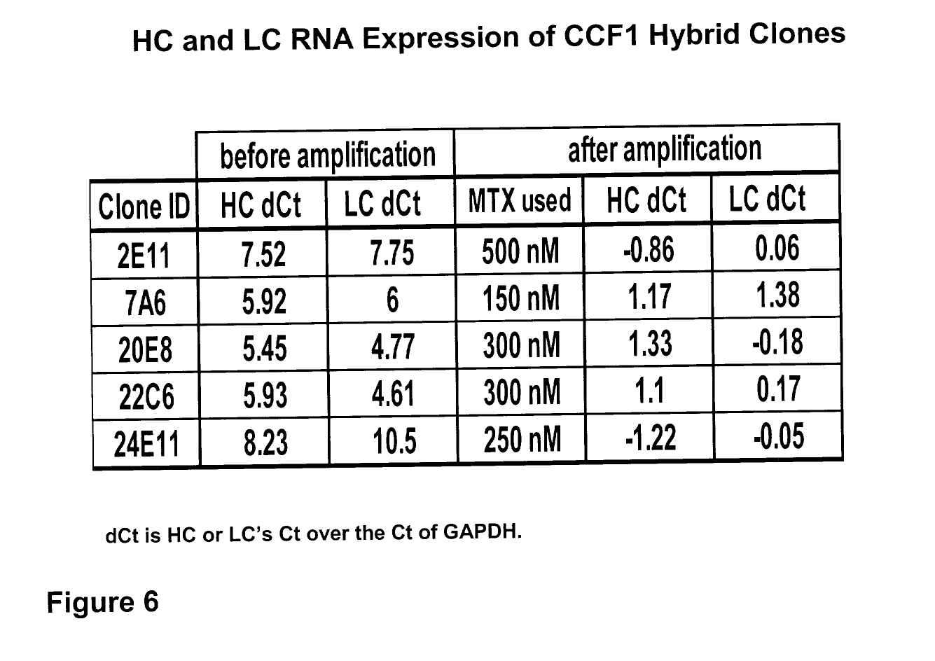

Single cell clones of MTX amplified hybrid cells were obtained by plating the MTX amplified cells with highest antibody titer in 96 well plates at appropriate cell densities in the MTX, Neo, and Hyg containing selection media to ensure that clones were originally from a single cell. Antibody titer of clones in 96 well plates was first determined by ELISA analysis of the culture supernatant. Antibody titers of the high producing clones were further ranked by ELISA analysis of the 4-day cell culture supernatant as described above. RNA expression of heavy chain and light chain of hybrid cells before and after DHFR amplification were also measured by quantitative PCR using Taqman. FIG. 6 shows the HC and LC expression level of five CCF1 clones before and after MTX amplification. RNA was isolated from cells before and after MTX amplification. HC RNA, LC RNA, as well as a normalization gene GAPDH RNA expressed in the cell were determined by quantitative PCR using an ABI Prism 7700 Sequence Detection System. The threshold cycle (Ct), which indicates the cycle number at which the amount of amplified target reaches a certain threshold, for HC, LC, and a normalization gene GAPDH were measured using method well known in the art. The higher the Ct, the lower the RNA level, i.e., more PCR cycles are required to amplify the target to reach the pre-set threshold. The relative HC and LC RNA expression level in the cell were indicated by their delta Ct (dCt), which is the result of the Ct value of HC or LC RNA minus the Ct value of GAPDH RNA level expressed in the same cell. The higher the dCt value, the lower the RNA level.

Analysis of the results showed that although heavy chain and light chain genes were located on different chromosomes in the hybrid cells, both heavy chain and light chain genes were amplified in all clones tested. Antibody production level of hybrid clones was greatly increased with DHFR amplification as shown in the table of FIG. 6.

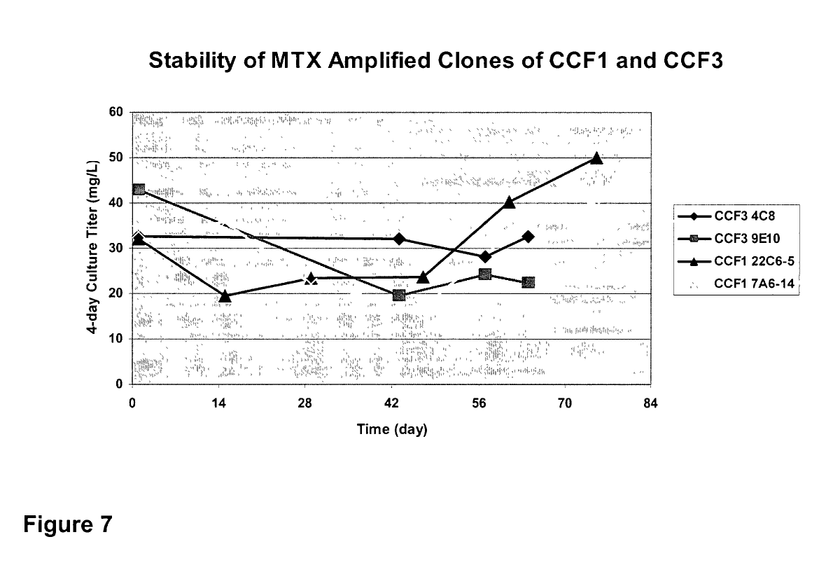

Clones that secreted the highest amounts of antibody were expanded in culture and adapted to suspension growth in spinner flasks or shaker flasks in the presence of MTX. The specific productivity of the clones was measured using method known in the art. The stability of the amplified hybrid clones was determined by measuring the antibody titers and specific productivity of the clones over a period of 2-3 months. Analysis of the results showed that highly productive and stable manufacturing cells for recombinant antibody production were generated using cell-cell fusion and post-fusion amplification of hybrid cells (FIG. 7).

Comparison of Antibody Production by Hybrid Cells Produced using the CCF1 Protocol, the CCF2 Protocol, or Pre-Fusion Gene Amplification on Parental Cells Prior to Fusion (“CCF3”)

Two clones each from CCF1 (22C6 and 7A6) and CCF2 (4-5F12 and 3-6B8) were amplified with various concentrations of MTX. In CCF3, heavy chain transfectant clone (18B11) and light chain transfectant clone (1G9) were both amplified with 250 nM MTX. The amplified pools of HC transfectant cells and LC transfectant cells were fused. Pools of hybrid cells were selected with Neo, Hyg and 0, 250, and 500 nM of MTX. Antibody titers of the three fused pools generated using the CCF3 protocol, as well as the amplified pools of the CCF2 protocols, were determined by Elisa analysis of the supernatant of a 4-day culture for complete antibody. The results are provided in the table in FIG. 5. These data show that post-amplification fusion is at least as good and, when selecting clone prior to fusion, better than pre-fusion amplification.

The instant invention shown and described herein is what is considered to be the most practical and the preferred embodiments. It is recognized, however, that departures may be made therefrom which are within the scope of the invention, and that obvious modifications will occur to one skilled in the art upon reading this disclosure.

What is claimed is:

1. A method for producing a multi-component protein, said method comprising:
(a) introducing a first polynucleotide into a first mammalian cell, wherein the first polynucleotide comprises a first amplifiable marker and a sequence encoding a first component of the multi-component protein, wherein the first amplifiable marker is amplifiable by an amplification agent;
(b) introducing a second polynucleotide into a second mammalian cell, wherein the second polynucleotide comprises a second amplifiable marker and a sequence encoding a second component of the multi-component protein, wherein the second amplifiable marker is amplifiable by the same amplification agent as the first amplifiable marker;
(c) optionally, repeating step (b) for each remaining component of the multi-component protein;
(d) fusing cells produced from steps (a)-(c) to form a hybrid cell, wherein the hybrid cell expresses the multi-component protein; and
(e) culturing the hybrid cell in the presence of the amplification agent for the first and second amplifiable markers.

2. The method of claim 1, further comprising:
(f) recovering the multi-component protein from the hybrid cell culture.

3. The method of claim 1, wherein the multi-component protein is an antibody.

4. The method of claim 1, wherein the first cell and the second cell are selected from the group consisting of a myeloma cell and a non-lymphoid cell.

5. The method of claim 1, wherein the first cell and the second cell are Chinese hamster ovary (CHO) cells.

6. The method of claim 5, wherein the CHO cells are DHFR negative.

7. The method of claim 1, wherein the first component is a heavy chain polypeptide, the second component is a light chain polypeptide, and the multi-component protein is an antibody.

8. The method of claim 1, wherein the first component is a heavy chain polypeptide, and the method further comprises identifying a first cell expressing a heavy chain polypeptide by detecting secreted heavy chain polypeptide.

9. The method of claim 1, wherein the first and second amplifiable markers are selected from the group consisting of dihydrofolate reductase (DHFR), glutamine synthase, and adenosine deaminase.

10. A method for producing an antibody, said method comprising:
fusing a first recombinant mammalian cell and a second recombinant mammalian cell to form a hybrid cell, wherein the first cell contains a first polynucleotide comprising a first amplifiable marker and a sequence encoding a heavy chain polypeptide, and wherein the second cell contains a second polynucleotide comprising a second amplifiable marker and a sequence encoding a light chain polypeptide, wherein the first and second amplifiable markers are amplifiable by the same amplification agent; and
culturing the hybrid cell in the presence of the amplification agent for the first and second amplifiable markers.

11. The method of claim 10, further comprising:
recovering the antibody from the hybrid cell culture.

12. The method of claim 10, wherein the first cell and the second cell are selected from the group consisting of a myeloma cell and a non-lymphoid cell.

13. The method of claim 10, wherein the first cell and the second cell are Chinese hamster ovary (CHO) cells.

14. The method of claim 10, wherein the first cell is a CHO cell, and the method further comprises identifying a first cell expressing a heavy chain polypeptide by detecting secreted heavy chain polypeptide.

15. The method of claim 10, wherein the polynucleotide encoding the heavy chain polypeptide and the polynucleotide encoding the light chain polypeptide are obtained from a B-cell or a hybridoma cell, wherein said B-cell or hybridoma cell produce an antibody.

16. The method of claim 10, wherein the first cell expresses an irrelevant light chain and expresses the desired heavy chain prior to fusion with the second cell.

17. The method of claim 10, wherein the first cell does not express a light chain polypeptide, and where the method further comprises, prior to said fusing, identifying the first cell by detection of heavy chain polypeptide secreted in culture supernatant of the first cell.

18. The method of claim 17, wherein the first cell is a CHO cell.

19. The method of claim 10, wherein the first cell expressing the desired heavy chain is selected for one or more desirable characteristics prior to said fusing.

20. The method of claim 10, wherein both the second cell expressing the desired light chain is selected for one or more desirable characteristics prior to said fusing.

21. The method of claim 19, wherein said desirable characteristic is selected from the group consisting of high production rate of the heavy chain.

22. The method of claim 20, wherein said desirable characteristic is selected from the group consisting of high production rate of the light chain.

23. The method of claim 10, wherein the first and second amplifiable markers are selected from the group consisting of dihydrofolate reductase (DHFR), glutamine synthase (GS), and adenosine deaminase.

24. A method for producing an antibody, said method comprising:
identifying a first recombinant mammalian cell secreting a heavy chain polypeptide, wherein the first mammalian cell contains a first polynucleotide comprising a first amplifiable marker and a sequence encoding a heavy chain polypeptide, wherein the first CHO cell does not express an irrelevant light chain polypeptide, said identifying being by detection of heavy chain polypeptide in culture supernatant of the first mammalian cell;
fusing a second recombinant mammalian cell to the identified first mammalian cell, the second mammalian cell containing a second polynucleotide comprising a second amplifiable marker and a sequence encoding a light chain polypeptide, said fusing producing a hybrid cell; and
culturing the hybrid cell in the presence of the amplification agent for the first and second amplifiable markers.

25. The method of claim 24, further comprising:
recovering the antibody from the hybrid cell culture.

26. The method of claim 25, wherein the first and second amplifiable markers are amplifiable by the same amplification agent.

27. The method of claim 25, wherein the first and second recombinant mammalian cells are not cultured in the presence of an amount of the amplification agents sufficient to provide for amplification of the amplifiable marker prior to said fusing.

28. The method of claim 24, wherein the polynucleotide encoding the heavy chain polypeptide and the polynucleotide encoding the light chain polypeptide are obtained from a B-cell or a hybridoma cell, wherein said B-cell or hybridoma cell produce an antibody.

29. The method of claim 24, wherein the first and second amplifiable markers are selected from the group consisting of dihydrofolate reductase (DHFR), glutamine synthase (GS), and adenosine deaminase.

30. A method for producing a multi-component protein, said method comprising:
(a) introducing a first polynucleotide into a first mammalian cell, wherein the first polynucleotide comprises a first amplifiable marker and a sequence encoding a first component of the multi-component protein, wherein the first amplifiable marker is amplifiable by an amplification agent, said introducing producing a first recombinant cell;
(b) introducing a second polynucleotide into a second mammalian cell, wherein the second polynucleotide comprises a second amplifiable marker and a sequence encoding a second component of the multi-component protein, wherein the second amplifiable marker is amplifiable by a second amplification agent, said introducing producing a second recombinant cell;
(c) optionally, repeating step (b) for each remaining component of the multi-component protein;
(d) fusing cells produced from steps (a)-(c) to form a hybrid cell, wherein the hybrid cell expresses the multi-component protein; and
(e) culturing the hybrid cell in the presence of the amplification agents for the first and second amplifiable markers;
wherein the said first and second recombinant not cultured in the presence of an amount of the amplification agents sufficient to provide for amplification of the amplifiable marker until after said fusing.

31. The method of claim 30, wherein the first and second amplifiable markers are amplified by different amplification agents.

32. The method of claim 30, wherein the first and second amplifiable markers are amplified by the same amplification agents.

33. The method of claim 30, further comprising:
(f) recovering the multi-component protein from the hybrid cell culture.

34. The method of claim 30, wherein the multi-component protein is an antibody.

35. The method of claim 30, wherein the first cell and the second cell are Chinese hamster ovary (CHO) cells.

36. The method of claim 35, wherein the CHO cells are DHFR negative.

37. The method of claim 30, wherein the first component is a heavy chain polypeptide, and the method further comprises identifying a first cell expressing a heavy chain polypeptide by detecting secreted heavy chain polypeptide.

38. A method for producing an antibody, said method comprising:
fusing a first recombinant mammalian cell and a second recombinant mammalian cell to form a hybrid cell, wherein the first cell contains a first polynucleotide comprising a first amplifiable marker and a sequence encoding a heavy chain polypeptide, and wherein the second cell contains a second polynucleotide comprising a second amplifiable marker and a sequence encoding a light chain polypeptide; and
culturing the hybrid cell in the presence of the amplification agent for the first and second amplifiable markers,
wherein the first and second recombinant not cultured in the presence of an amount of the amplification agents sufficient to provide for amplification of the amplifiable marker prior to said fusing.

39. The method of claim 38, further comprising:
recovering the antibody from the hybrid cell culture.

40. The method of claim 38, wherein the first cell and the second cell are Chinese hamster ovary (CHO) cells.

41. The method of claim 38, wherein the first cell does not express a light chain polypeptide, and where the method further comprises, prior to said fusing, identifying the first cell by detection of heavy chain polypeptide secreted in culture supernatant of the first cell.
